(19) United States
(12) Patent Application Publication
(10) Pub. No.: US 2006/0084934 A1
(43) Pub. Date: Apr. 20, 2006

(54) Transponder assembly and method for making same

(76) Inventor: Milton Frank, Hackensack, NJ (US)

Correspondence Address: EPSTEIN DRANGEL BAZERMAN & JAMES, LLP, 60 EAST 42ND STREET, SUITE 820, NEW YORK, NY, 10165, US

(21) Appl. No.: 10/966,434

(22) Filed: Oct. 18, 2004

(51) Int. Cl. A61F 13/15

(52) U.S. Cl. 604/362

(57) ABSTRACT

A transponder assembly for use in identifying surgical implements such as sponges comprises a transponder substantially encased in a plastic body to which a base having an exposed adhesive surface is attached. The assembly may be fastened directly to a rigid implement or may be securely attached to a sponge by a supporting body embodying a pin-head clutch.

### FIELD OF THE INVENTION

The present invention relates to a transponder particularly designed to identify individual surgical sponges or implements, and to a method for making it.

### BACKGROUND OF THE INVENTION

Surgical operations involve the use of implements and blood-absorbing sponges which are placed within the wound necessarily created by the operation, there to remain for a period of time during the operation before being removed. Obviously it is essential that all such objects be removed from the patient's body before the incision is closed. If a sponge, clamp or other implement is left behind in the patient's body it will be a source of infection and trouble, potentially fatal. Accordingly one major task in any operation is to keep track of the sponges and implements used so as to make sure that the operation is terminated in an effective and medically wholesome manner. Over the years many proposals have been made for accomplishing this desired result with as high a degree of certainty as possible.

One such general approach is to so modify the sponges or implements used as to make them radiation-sensitive—receiving radiation directed at them and reacting in some manner capable of being sensed by appropriate equipment so as to indicate their presence in the patient's body, thus enabling the operating team to interrogate by means of radiation and to receive some signal indicating that a foreign object is present. More specifically, it has been proposed that each implement, be it sponge, clamp or whatever, that is placed within the patient's body, thus identify itself with a signal unique to each implement, such as by a unique serial number or the like. In this way the operating team, using available circuitry and computers, can record the identification of each item placed within the patient's body, can later record the identification of each item removed from the patient's body, and can then compare the lists of identifications to determine whether something is unaccounted for, and if it is what it is, thereby to facilitate the finding and retrieval of the foreign object in question.

Known devices, called transponders, are capable of receiving a transmitting radiation signal and of transmitting in response their own unique identification radiation signal. However, means must be provided for the transponders to be attached to such disparate objects as surgical sponges, clamps and other surgical instruments in such a manner that they remain with such items throughout their use during the operation, since leaving a transponder behind would in all likelihood be just as dangerous as leaving a sponge behind. The problem is intensified by the fact that a given operation will involve the use of a large number of items that must be kept track of.

### SUMMARY OF THE INVENTION

In accordance with the present invention, a transponder assembly is produced which is suitable for relatively inexpensive quantity production, which is readily attachable to such diverse surgical impedimenta as fabric sponges and metal implements, and which will reliably remain in position once appropriately attached. In such an assembly the transponders are initially substantially completely encased within a plastic body preferably having significant adhesive properties, and a base is secured to that plastic body. An exposed surface of that base is itself adhesive in character, as by applying to the plastic body a double-faced adhesive strip. In certain circumstances the double-faced adhesive strip may constitute the base, but in other circumstances the base may comprise an additional part, such as a thin steel piece, to which the double-sided adhesive strip is attached.

The exposed adhesive surface on the base may be applied directly to a rigid tool such as a clamp, thereby to reliably secure the transponder in place of that tool, but to attach the transponder to a flexible fabric such as a surgical sponge is another matter. For that purpose a supporting body is provided for each transponder to an exposed surface of which the transponder assembly is adhesively fastened. That supporting body has a pin extending therefrom which is adapted to penetrate and project from the sponge, and a retaining element is frictionally fastened to the extending portion of the pin, this being the well-known pin-clutch assembly.

Large numbers of transponder assemblies of the present invention will be required since significant numbers of items, particularly sponges, are placed within the patient's body during a typical operation. The particular construction of the transponder assembly of the present invention is well suited to relatively inexpensive quantity production. The transponder unit itself is embedded within and essentially completely surrounded by a self-sustaining plastic mass, preferably inherently of adhesive character but at any rate having an exposed surface which is, inherently or otherwise, of adhesive character. A base is secured to the transponder, the exposed surface of that base exhibiting adhesive properties. The base itself may be constituted by a double-sided adhesive strip or, if additional structural strength is required, may comprise two layers, the first layer, adhesively secured to the transponder-plastic combination, being structural in character such as a metal strip, with the second layer preferably being the aforementioned double-sided adhesive strip. The thus-constructed transponder assembly may be very conveniently manufactured by means of an open-topped mold preferably in the form of a flexible sheet having multiple open-topped cavities of appropriate size and shape. A transponder and an appropriate quantity of unset plastic material is inserted into each open-topped cavity, the desired base is applied thereto, the plastic mass is caused to set, and then the individual transponder assemblies can be removed from their respective cavities merely by flexing the mold sheet. In order to ensure that the plastic material properly engages and protects the transponder with which it is associated, it is preferred that the unset plastic material is inserted into a mold cavity in two stages, a preliminary amount first being placed in the cavity, the transducer then being pushed into that plastic, and a second quantity of plastic then being added to fill the cavity and preferably substantially completely cover the transducer, before the base is applied.

Thus the transponders are substantially insulated and protected from external problems while at the same time are readily manipulatable and capable of reliably functioning in the external environment to which they will be subjected in the course of a surgical operation.

### BRIEF DESCRIPTION OF THE DRAWINGS

To the accomplishment of the above, and to such other objects as may hereinafter appear, the present invention relates to the construction of a transponder assembly and the method of making same as defined in the appended claims and as described in this specification and disclosed in the following drawings:

FIG. 1 is a perspective exploded view of the attachment of the transponder assembly of the present invention to a surgical sponge;

FIG. 2 is an exploded view of one embodiment of the transponder assembly of the present invention;

FIG. 3 is an exploded view of a second embodiment of the transponder assembly of the present invention;

FIG. 4 is a three-quarter exploded view of that part of the assembly of FIG. 1 that carries the sponge-engaging pin;

FIG. 5 is a three-quarter view showing a first step in the preferred method of formation of the assembly of the present invention;

FIGS. 6, 7, 8 and 9 are three-quarter views similar to FIG. 5 but showing successive steps in the preferred formation of the transponder assembly;

FIG. 10 is a three-quarter perspective fragmentary view of a typical mold sheet that may be used in the preferred formation method, with the individual mold cavities filled with assemblies of FIG. 2;

FIG. 11 is a cross-sectional view taken along the line 11-11 of FIG. 10; and

FIG. 12 is a view similar to FIG. 11 but showing transponder assemblies of FIG. 3 in the mold cavities.

### DESCRIPTION OF THE PREFERRED EMBODIMENTS

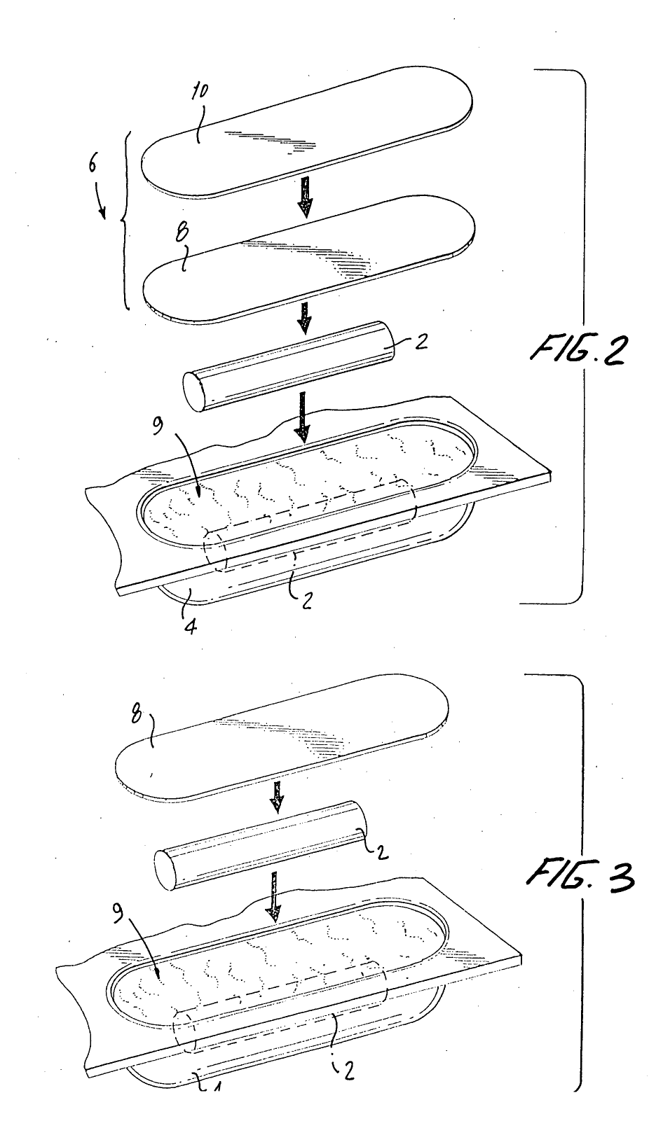

Transponder—devices capable of receiving a radiation signal and transmitting a radiation signal in response—are readily available in sizes capable of being used in connection with this invention. The basic transponder assembly of the present invention comprises a transponder 2, here shown as a small cylinder, embedded in and substantially covered by a plastic mass 4 attached to a base 6 the exposed surface of which is adhesive in character. The base 6 preferably comprises a double-faced adhesive strip 8 secured either directly or indirectly to an exposed surface of plastic mass 4. If desired for improved structural reliability, and as shown in FIG. 2, a strip 10 of suitable structural material such as metal may be interposed between the adhesive strip 8 and the plastic mass 4, being secured to the plastic. Alternatively, and as illustrated in FIG. 3, the metal strip 10 may be omitted, the adhesive strip 8 being applied directly to the plastic mass 4.

These individual assemblies may readily be formed on a quantity basis by utilizing a mold sheet 12 having a plurality of appropriately sized and shaped open-topped cavities 14. The mold sheet 12 is preferably formed of flexible material to facilitate ejection of the assemblies from the sheet. A typical sheet may have a hundred rows of a hundred cavities 14 each.

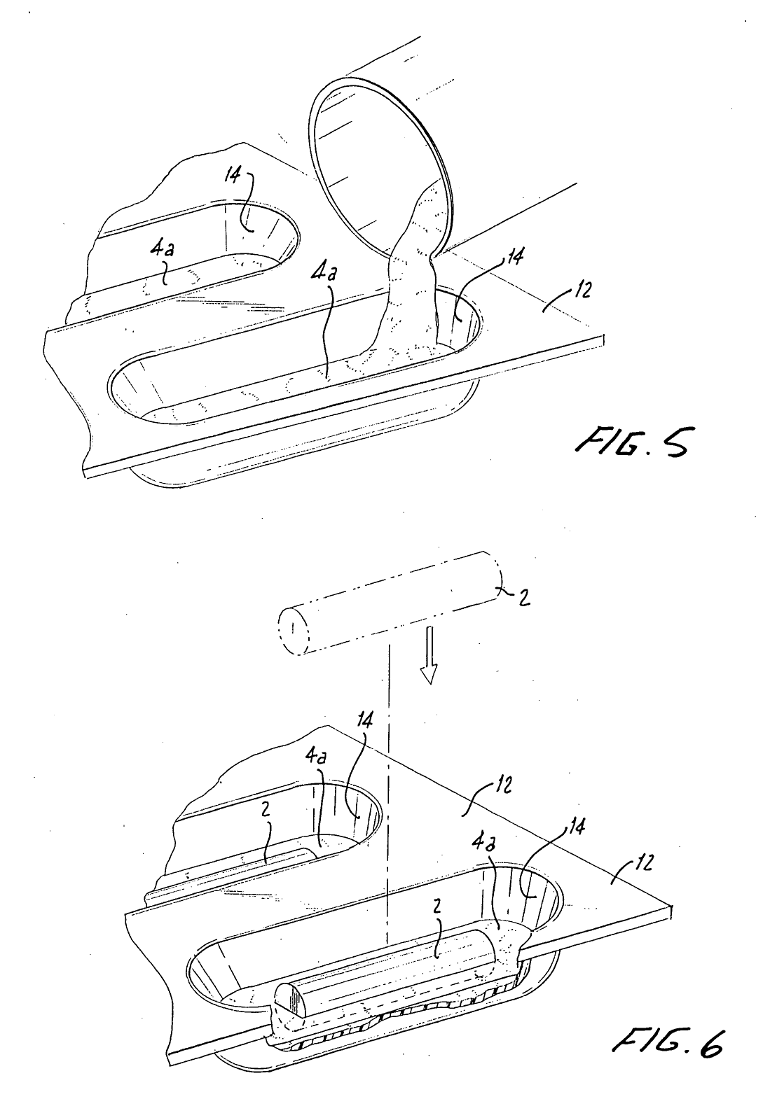

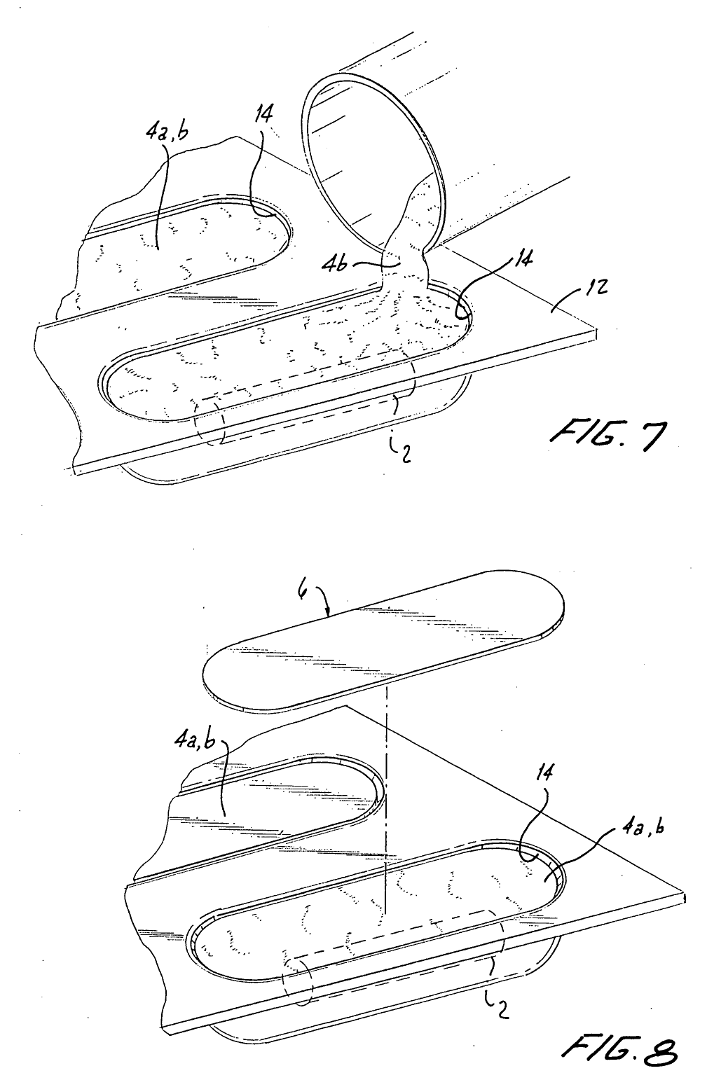

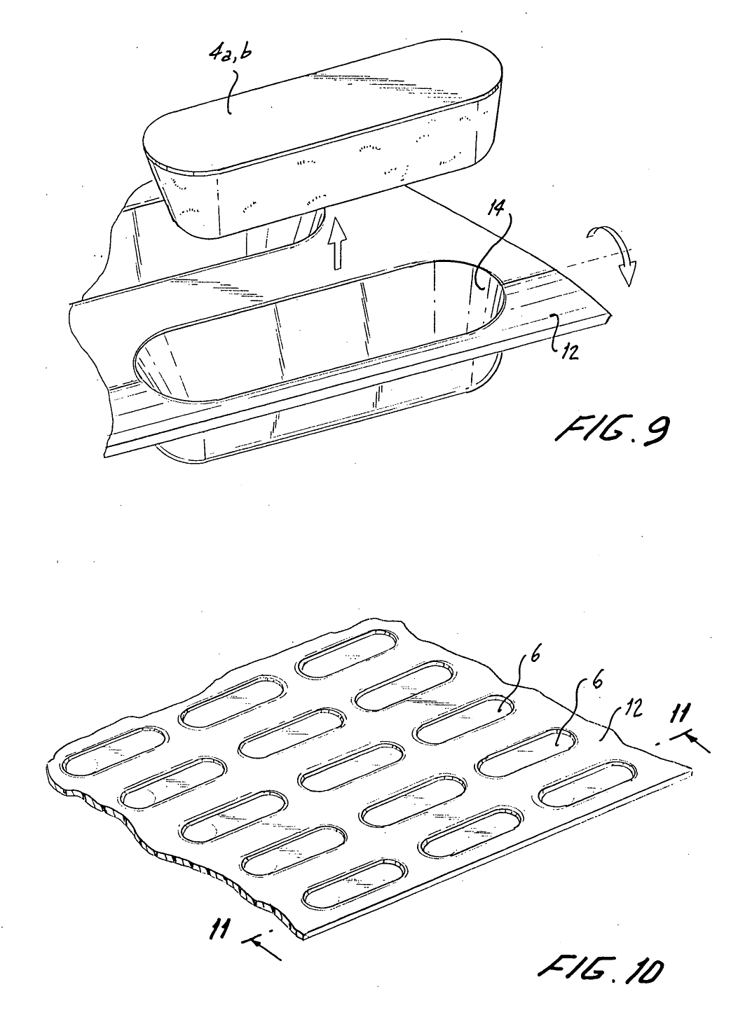

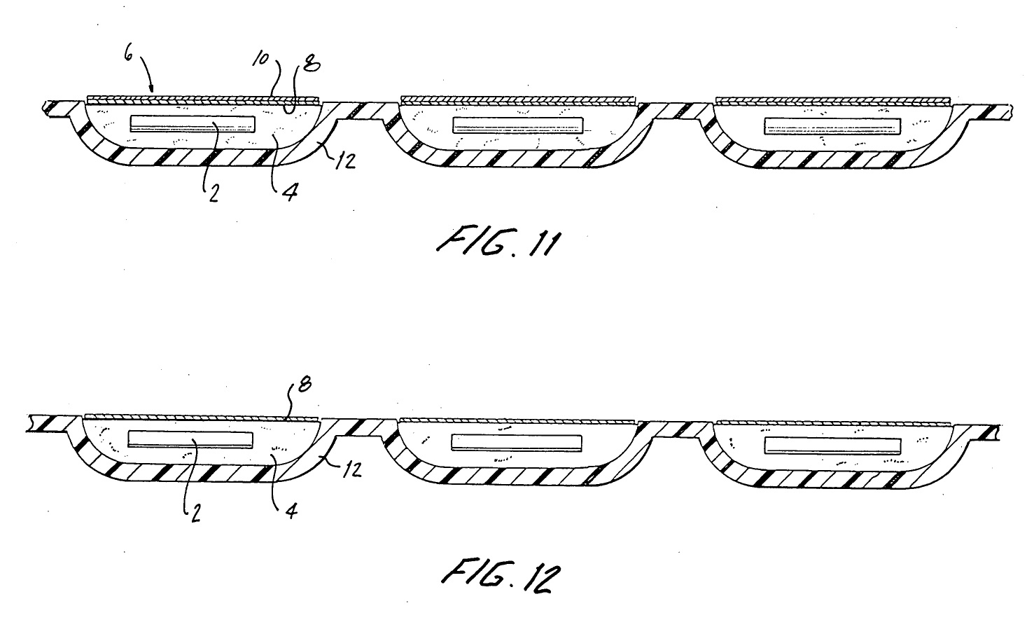

As illustrated in FIG. 5, the first step in the preferred method of fabrication of the transponder assembly 2 is to place, usually by pouring, a first quantity 4a of that plastic material into a cavity 14 so as to partially but not completely fill that cavity. Next, as shown in FIG. 6, a transponder 2 is inserted into the plastic mass 4a in a cavity 14 so as to be at least partially submerged, this procedure tending to insure that there is a layer of the plastic material 4a between the transponder 2 and the bottom of the cavity 14. Next, as shown in FIG. 2, a second mass of plastic material 4b is poured or otherwise placed within the cavity 14 to substantially fill that cavity and substantially completely cover the transponder 2. Next, as shown in FIG. 8, the assembly's base 6 may be placed on top of the unset plastic mass 4, preferably fitting relatively snugly within the cavity 14 so as to effectively close the open top thereof. As indicated above, the base 6 may be constituted by a double-sided adhesive sheet of conventional construction the lower and upper surfaces of which, as the name implies, exhibit adhesive properties. Alternatively and as shown in FIG. 2, the base 6 may be in two parts, a structurally strong strip 10 of metal such as steel lying directly on the plastic mass 4 with the double sided adhesive strip 8 adhesively secured to the upper surface of the strip 10.

The substance used for the plastic mass 4 is one which can readily be inserted into the cavity 14, as by pouring, yet in the finished product will have form, solidity and shape so as to carry and protect the transponder 2. Thus a thermosetting resin is preferred for this purpose. Moreover, it is desirable that the material 4 when set itself have adhesive properties. When the base 6 comprises the metal strip 10 it is preferable that the strip 10 is applied to the plastic mass 4 before the latter has set, particularly when the mass 4 itself is adhesive, so that adhesion of the strip 6 with the remainder of the assembly will be reliably and permanently obtained. However, if needed, a separate adhesive layer may be interposed between the metal strip 6 and the plastic 4. The double-sided adhesive strip 8 whether used with or without the metal strip 10 may be applied to the assembly either before or after the plastic mass 4 has set. I prefer, when the metal strip 10 is used, to apply the adhesive strip 8 to the metal strip 10 to form the base 6 and then apply the thus-formed base 6 to the plastic mass 4 before the latter is subjected to setting conditions.

I have found that epoxy adhesives, and particularly those of the room temperature curable type, are very effective. I have used such epoxy adhesives as those sold by Loctite under the designations M-31CL, 3981, E-20EP and E-120HP, as well as the epoxy adhesives sold by MasterBond under the designations EP42HT and EP42HT-2. For double-sided adhesive strips I have used those sold by 3M under the designations 468MP and 9469-PC.

After the cavities 14 have been filled as thus described the sheets 12 may then be subjected to such external conditions as will cause the particular material 4 to set. With the materials specified above, letting the filled trays stand at room temperature for twenty-four hours provides the desired setting condition.

After the plastic material 4 in the cavity 14 has set the individual transponder assemblies can readily be removed from the sheet 12 because all of the cavities 14 have open tops. One convenient way in which the assemblies may thus be removed is by flexing the sheet 12, causing the individual transponder assemblies to pop out from their respective cavities 14 in the same way that ice cubes may be popped out of flexible ice cube trays.

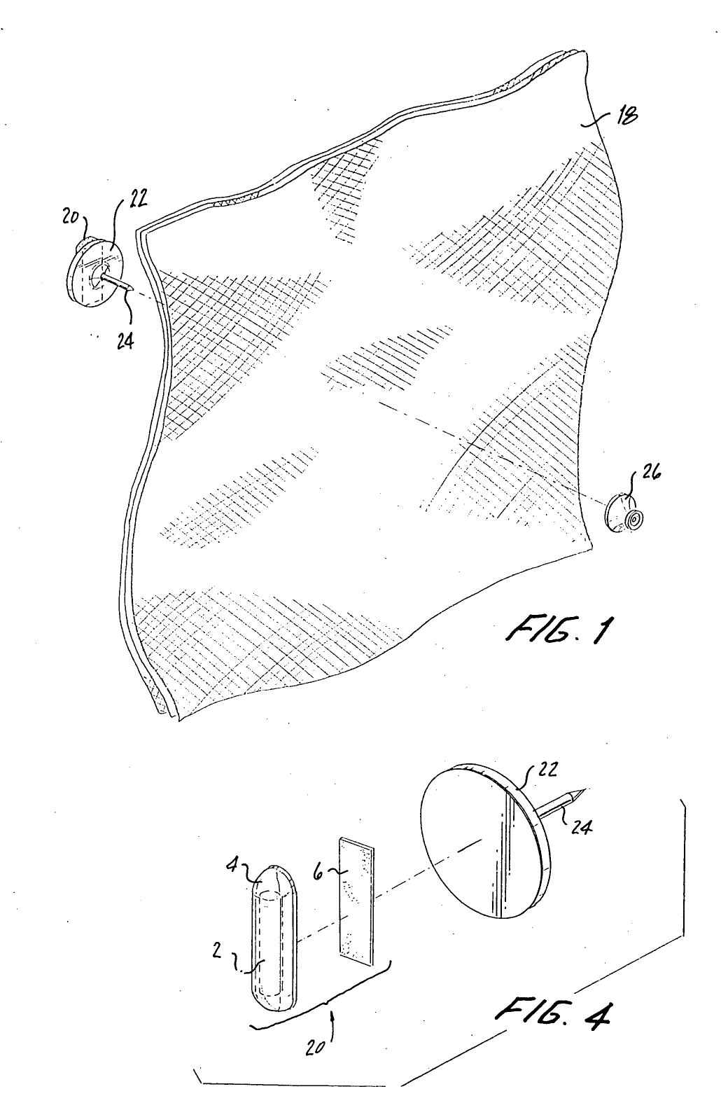

The exposed adhesive surface of the double-sided adhesive strip functions satisfactorily when attaching the assemblies to a rigid operating tool such as a clamp. However, the flexible and usually loosely-woven material of a surgical sponge 18, more or less schematically shown in FIG. 1, makes impractical the adhesive attachment of the assemblies of the present invention directly to the sponge. Therefore, as shown in FIGS. 1 and 4, the complete transponder assembly 20 is attached to a supporting body 22 in the form of a preferably metal disc by means of its exposed adhesive surface, here shown as constituted by the exposed surface of the double-sided adhesive strip 8. Projecting from the opposite surface of the supporting body 22 is a pin 24 designed to penetrate and extend beyond the body of the sponge 18, and a retaining element 26 is designed to slide over the pin 24 and resist removal therefrom. This type of device is well-known, and often designated a pin-head clutch. The action of this type of device to secure a transponder assembly 20 to a sponge 18 has the advantage that the same transponder assembly 2 with pin-head clutch 22, 26 may be removed from the sponge 18 to which it is connected before that sponge is discarded, with the assembly 20, 22, 26 being ready for use with another sponge 18 as is required.

With the present arrangement readily available transponders can be adapted to be secured to virtually any type of implement and device used in connection with surgical operations so as to perform vitally needed identification functions. The resulting assemblies may be manufactured in a simple, reliable and relatively inexpensive manner, and when made will protect the transponder while making it readily manipulatable and effectively operable.

While only a limited number of embodiments have been here specifically disclosed, it will be apparent that many variations may be made therein, all within the scope of the invention as defined in the following claims.

I claim:

1. A transponder assembly comprising a base having upper and lower surfaces, a transponder operatively mounted on the upper surface of said base, a plastic body secured to said base and engaging and at least partially covering said transponder and securing said transponder to said base, said base having an exposed lower surface to which an adhesive may be applied.

2. A transponder assembly comprising a base having upper and lower surfaces, a transponder operatively mounted on the upper surface of said base, a plastic body secured to said base and engaging and at least partially covering said transponder and securing said transponder to said base, and an adhesive layer operatively exposed at the lower surface of said base.

3. The transponder assembly of claim 2, in which said base comprises two layers, the first layer comprising a metal plate attached to said plastic body and said second layer comprising said exposed adhesive layer attached to said plate.

4. The transponder assembly of either of claims 2 or 3, in which said adhesive layer comprises a supportive sheet with adhesive on both sides, the adhesive on one side attaching said sheet to the remainder of said assembly and the adhesive on the other side constituting said operatively exposed adhesive layer.

5. A transponder assembly adapted to be secured to a surgical sponge or the like comprising a supporting body having first and second sides with a pin extending from said first side thereof, said pin being adapted to penetrate a sponge with said body on one side of said sponge and said pin extending from the other side of said sponge, a retaining element adapted to engage the extended portion of said pin, thus securing said body to said sponge, and a transponder secured to said supporting body at said second side of said body.

6. A transponder assembly adapted to be secured to a surgical sponge or the like comprising a supporting body with a pin extending therefrom, said pin being adapted to penetrate a sponge with said body on one side of said sponge and said pin extending from the other side thereof, a retaining element adapted to engage the extended portion of said pin, thus securing said body to said sponge, and the transponder assembly of either of claims 2 or 3 secured by its exposed adhesive layer to said supporting body.

7. The method of making a transponder assembly comprising placing into an open-topped mold cavity a transponder and an amount of unset settable material, adding an additional amount of said material to said cavity if needed so as to at least substantially cover the transponder with said material, curing said settable material, and removing said set article from said mold cavity.

8. In the method of claim 7, securing to the upper surface of said article a base having an exposed surface to which an adhesive may be affixed.

9. The method of claim 8, in which said base comprises two layers secured to one another, the first layer comprising a metal plate engaging said article and said second layer comprising an adhesive layer exposed on said first layer.

10. The method of claim 9, in which said base is applied to said article before said material is set.

11. The method of claim 9, in which said base is applied to said article after said material is set.

12. The method of claim 8, in which said adhesive comprises a sheet of material with adhesive on both sides, the adhesive on one side attaching said sheet to said article and the adhesive on the other side being operatively exposed.

13. The method of claim 12, in which said sheet is applied to said article before said material is set.

14. The method of claim 12, in which said sheet is applied to said article after said material is set.

15. The method of any of claims 7-14, in which a plurality of said open-topped mold cavities formed in a flexible sheet are utilized to respectively mold said transponder and said settable material, and in which said set articles are removed from their respective mold cavities by flexing said sheet.

16. The method of any of claims 7-14, in which settable material is first placed into said mold cavity and said transponder is thereafter placed into said settable material.

17. The transponder assembly of either of claims 2 or 3, in which said plastic body comprises an adhesive substance.

18. The transponder assembly of claim 4, in which said plastic body comprises an adhesive substance.

19. The transponder assembly of either of claims 2 or 3, in which said plastic body comprises an epoxy adhesive.

20. The transponder assembly of claim 4, in which said plastic body comprises an epoxy adhesive.

21. The method of any of claims 7-9, in which said settable material comprises an adhesive substance.

22. The method of any of claims 7-9, in which sais settable material comprises an epoxy adhesive.
